(19) United States
(12) Patent Application Publication
(10) Pub. No.: US 2019/0166884 A1
(43) Pub. Date: Jun. 6, 2019

(54) Isoflavone-Supplemented Chick Diets and Methods of Feeding the Same

(71) Applicant: PURINA ANIMAL NUTRITION LLC, Shoreview, MN (US)

(72) Inventors: Bill L. Miller, Labadie, MO (US); James D. Caldwell, St. Clair, MO (US)

(21) Appl. No.: 15/833,744

(22) Filed: Dec. 6, 2017

(51) Int. Cl. A23K 50/75; A23K 20/10; A23K 10/30; A61K 31/352

(52) U.S. Cl. CPC A23K 50/75; A61K 31/352; A23K 10/30; A23K 20/10

(57) ABSTRACT

Isoflavone-supplemented dry feed fed to young poultry animals provides total levels of isoflavones in the feed at about 1640 mg/kg feed to about 3890 mg/kg feed, and/or provides an additional about 750 mg/kg feed to about 3000 mg/kg feed. In response to ingesting the isoflavone-supplemented feed, the young poultry animal experiences improved performance.

### TECHNICAL FIELD

The present disclosure relates generally to feed for young poultry supplemented with isoflavones and systems and methods of feeding young poultry such feed.

### BACKGROUND

The time it takes poultry animals to mature to a stage where the animal can produce meat or eggs is important in the poultry commodity market. A number of feeding systems have been used to enhance weight gain of poultry beginning at a young age. Such techniques may involve providing feed supplemented with vitamins, minerals, medication, and other components that may benefit the young animals. This may, for example, reduce the point-of-lay age in pullets, thereby reducing the cost of egg production. Increased weight gain of poultry, such as broiler chickens, from an early age may also reduce the cost of meat production.

### SUMMARY

Some implementations provide methods of feeding young poultry animals an isoflavone-supplemented feed. In embodiments, a method of feeding a young poultry animal an isoflavone-supplemented feed includes providing a feed to a young poultry animal for at least one week during the first three weeks of the young poultry animal's life. The young poultry animal ingests the feed and experiences improved performance in response to ingesting the feed. The feed includes a supplemental level of isoflavone added to the feed such that the total isoflavone level is about 1640 mg/kg of the feed to about 3890 mg/kg of the feed.

In embodiments, the feed is provided to the young poultry animal is for at least about three weeks and at least one week is during the first three weeks of the young poultry animal's life.

In embodiments, the feed includes protein at about 20 weight percent.

In embodiments, the isoflavone level of the feed prior to supplementation is about 890 mg/kg of the feed.

In embodiments, the supplemental isoflavone level is about 750 mg/kg of the feed to about 3000 mg/kg of the feed. The supplemental isoflavone level may be about 1500 mg/kg of the feed.

In embodiments, the improved performance experienced by the young poultry animal is one or both of an improved weight gain or an improved feed-to-gain ratio.

In embodiments, the young poultry animal is a broiler chick.

In embodiments, the isoflavone supplement is an isoflavone isolate derived from soy. The isoflavone supplement may include isoflavone aglycone. The isoflavone aglycone may include genistein aglycone.

In embodiments, a method of improving performance of a young poultry animal is disclosed. A feed is provided to a young poultry animal for at least one week during the first three weeks of the young poultry animal's life. The animal ingests the feed, which includes a supplemental level of isoflavone added to the feed such that the total isoflavone level is about 1640 mg/kg of the feed to about 3890 mg/kg of the feed. In response to ingesting the isoflavone-supplemented feed for the at least one week, the young poultry animal experiences improved performance compared to a young poultry animal that ingests the same feed without supplemental isoflavone.

In embodiments, the improved performance experienced by the young poultry animal is improved weight gain.

In embodiments, the improved performance experienced by the young poultry animal is improved feed-to-gain ratio.

In embodiments, the feed is provided to the young poultry animal is for at least about three weeks and at least one week is during the first three weeks of the young poultry animal's life.

In embodiments, the feed includes protein at about 20 weight percent.

In embodiments, the isoflavone level of the feed prior to supplementation is about 890 mg/kg of the feed.

In embodiments, the supplemental isoflavone level is about 750 mg/kg of the feed to about 3000 mg/kg of the feed.

In embodiments, the young poultry animal is a broiler chick.

### DETAILED DESCRIPTION

Isoflavones are plant-based, water-soluble plant flavonoids that can have a phytoestrogen effect, meaning that isoflavones can have effects on the body similar to estrogen due to the ability of isoflavones to bind to estrogen receptors. Isoflavones may sometimes be referred to as phytoestrogen isoflavones. Much research has been conducted to better understand the concerns and benefits of human consumption of isoflavones. Several benefits to adolescent and adult humans have been attributed to isoflavone supplementation. These benefits include cancer inhibition and reduction of issues associated with menopause (bone density, heat flashes, and depression). However, the effects of consuming isoflavones may be different, even contrary to that of estrogen. The effects of isoflavone consumption may also be species specific.

Implementations herein provide for isoflavone-supplemented dry feed and systems and methods of feeding the dry feed to young poultry animals. These may be healthy young animals. Young poultry animals that may ingest the isoflavone-supplemented dry feed include but are not limited to young chicks, ducks, geese, and turkeys. Animal studies assessing the merit of supplementing isoflavones in poultry dry feed were conducted by the inventors and the results are reported herein. The findings were significant and unexpected. Particularly, it has been discovered that young poultry improve performance when fed supplemental levels of isoflavones via dry feed.

The isoflavones of the present disclosure may be sourced from soy, red clover, other plants, and combinations of these. Isoflavones include but are not limited to glycitein, genistein, daidzein, biochanin, and formonenetin. Soybeans contain high levels of isoflavones, particularly glycitein, genistein, and daidzein. Red clover contains genistein, daidzein, biochanin (a genistein derivative), and formonenetin (a daidzein derivative). These isoflavones can be present in at least two forms: as a glycoside (e.g., glucoside or glucuronide) or as an aglycone. For isoflavone glycoside, the isoflavone is attached to a glycone, which is a sugar, and may be present in soy products such as raw soy products. For isoflavone aglycone, the isoflavone is present without any glycone attached and may be referred to as a free isoflavone. Isoflavone aglycone may be present in soy products such as fermented soy products. Genistein aglycone may be the predominant bioactive form of the isoflavones.

Isoflavones, both glycosides and aglycones, may be isolated from their source plants as an isolate. Isoflavones may be available in powder form and may be mixed with feed, such as started feed.

In some implementations, the isoflavone supplement may contain one or a combination of glycitein, genistein, daidzein, biochanin, and formonenetin. In some implementations, one or more isoflavones may be isolated from other isoflavones and provided as the isoflavone supplement to the feed of the present disclosure. For instance, genistein aglycone derived from soy may be isolated from other soy-containing isoflavones, such as glycitein and/or daidzein, and may be provided as the supplement.

In another example, the isoflavone supplement may contain a mixture of genistein, daidzein, and glycitein, and the supplement may be derived from soy. In this example, genistein may account for about 51 percent of the isoflavones, daidzein may account for about 36 percent of the isoflavones, and glycitein may account for about 13 percent of the total isoflavones in the supplement. In other examples, the isoflavones in the supplement may be provided in different ratios or combinations. For instance, genistein, as a glycoside or aglycone, may be provided alone or in combination with daidzein or glycitein as a glycoside or aglycone. In this example, genistein may account for 1 to 100 percent, 1 to 90, 1 to 80, 1 to 70, 1 to 60, 1 to 50, 1 to 40, 1 to 30, 1 to 20, or 1 to 10 percent of the isoflavone, with any balance being provided by daidzein, glycitein, or both. Alternatively, daidzein or glycitein may be provided in the preceding amounts, with any balance being provided by genistein or the other of the daidzein or glycitein, or a combination.

The amount of isoflavone used to supplement the dry feed of the present disclosure may be an amount that is effective to improve performance of the young poultry animal. In some implementations, this amount may be at least about 700 mg/kg of feed and up to about 4000 mg/kg of feed. In some implementations, the effective amount may be about 1500 mg/kg of feed. In some examples the amount of isoflavone supplement may be about 700 mg/kg to about 3500 mg/kg, about 700 mg/kg to about 3000 mg/kg, about 700 mg/kg to about 2500 mg/kg, about 700 mg/kg to about 2000 mg/kg, about 700 mg/kg to about 1500 mg/kg, about 700 mg/kg to about 1000 mg/kg, about 750 mg/kg to about 4000 mg/kg, about 1000 mg/kg to about 4000 mg/kg, about 1500 mg/kg to about 4000 mg/kg, about 2000 mg/kg to about 4000 mg/kg, about 2500 mg/kg to about 4000 mg/kg, about 3000 mg/kg to about 4000 mg/kg, about 750 mg/kg to about 3000 mg/kg, about 750 mg/kg to about to 1500 mg/kg, about 1500 mg/kg to about 2250 mg/kg, about 1500 mg/kg to about 3000 mg/kg, about 2250 mg/kg to about 3750 mg/kg, about 750 mg/kg, about 1500 mg/kg, about 2250 mg/kg, about 3000 mg/kg, or about 3750 mg/kg of feed.

After supplementation, the total amount of isoflavone in the feed of the present disclosure may be an amount that is effective to improve performance of the young poultry animal. In some implementations, this total amount may be at least about 1500 mg/kg of feed and up to about 5000 mg/kg of feed. In some implementations, the effective amount may be about 2390 mg/kg of feed. In some examples the total amount of isoflavone in the feed may be about 1500 mg/kg to about 4500 mg/kg, about 1500 mg/kg to about 4000 mg/kg, about 1500 mg/kg to about 3500 mg/kg, about 1500 mg/kg to about 3000 mg/kg, about 1500 mg/kg to about 2500 mg/kg, about 1500 mg/kg to about 2000 mg/kg, about 2000 mg/kg to about 5000 mg/kg, about 2500 mg/kg to about 5000 mg/kg, about 3000 mg/kg to about 5000 mg/kg, about 3500 mg/kg to about 5000 mg/kg, about 4000 mg/kg to about 5000 mg/kg, about 2000 mg/kg to about 4500 mg/kg, about 1640 mg/kg to about 3890 mg/kg, about 1640 mg/kg to about 2390 mg/kg, about 2390 mg/kg to about 3890 mg/kg, about 1640 mg/kg, about 2390 mg/kg, or about 3890 mg/kg of feed.

In some approaches, the young animal may ingest at least about 30 mg and up to about 300 mg of the supplemental isoflavone per day when fed ad libitum. In some examples, the young animal may ingest about 50 mg to about 300 mg, about 75 mg to about 300 mg, about 100 mg to about 300 mg, about 150 mg to about 300 mg, about 200 mg to about 300 mg, about 30 mg to about 250 mg, about 30 mg to about 200 mg, about 30 mg to about 150 mg, about 30 mg to about 100 mg, about 40 mg to about 250 mg, or about 50 mg to about 175 mg of isoflavone supplement per day.

In some approaches, the young animal may ingest at least about 70 mg and up to about 400 mg of total isoflavone per day when fed ad libitum. In some examples, the young animal may ingest about 100 mg to about 400 mg, about 150 mg to about 400 mg, about 200 mg to about 400 mg, about 250 mg to about 400 mg, about 300 mg to about 400 mg, about 70 mg to about 350 mg, about 70 mg to about 300 mg, about 70 mg to about 250 mg, about 70 mg to about 200 mg, about 70 mg to about 150 mg, about 90 mg to about 300 mg, about 125 mg to about 225 mg of total isoflavone per day.

Isoflavone-supplemented dry feed may be a solid feed and contain protein at a level of about 20 weight percent, such as about 18 to about 25 weight percent. Protein sources may be from soybean or grains, such as distillers grain or corn. Soybean-derived protein includes hydrolyzed soy protein modified, soy protein concentrate, and soy protein isolate. Because soybeans naturally contain isoflavones, some isoflavones may be present in feed that derives a portion of the nutrients from soy. However, a portion of the naturally present isoflavones may be washed out during processing of soy into a form where it is usable for a feed. For instance, soy protein concentrate is prepared from dehulled soybean seeds by removing most of the oil and water-soluble non-protein constituents, including isoflavones, such as through alcohol extraction, resulting in insubstantial levels of isoflavones being present in soy protein concentrate, e.g. about 0.047 mg/g or less of soy protein concentrate. Soy protein isolate generally includes similar levels of isoflavones compared to soy protein concentrate. Hydrolyzed soy protein modified is produced using techniques that allow a substantial portion of the original isoflavones to be retained, e.g., approximately 3.65 mg/g of hydrolyzed soy protein modified. Soybean meal, which may be processed into soy flour and added to a feed, retains much of its native isoflavones and generally contains about 3.41 mg/g of the soybean meal. Thus, relatively higher levels of isoflavones may be contained in feeds containing these forms of soy protein. Consequently, implementations herein are directed to isoflavone-supplemented feed in which the level of isoflavones present in the milk replacer is a supplemental level where isoflavones have been added to the feed regardless of the source of protein or other nutrients. Supplemental levels of isoflavones are above that which could naturally be present when a portion or all of a nutrient or nutrients in the feed is derived from soy.

Isoflavone-supplemented dry feed may contain fat at a level of about 3 weight percent to about 4 weight percent, such as about 3.5 weight percent. Dimethylamine may be present in the isoflavone-supplemented dry feed at about 11 weight percent to about 15 weight percent, such as about 12.7 weight percent. Isoflavone-supplemented dry feed may contain grain byproducts at about 6 weight percent to about 10 weight percent, such as about 8 weight percent. Molasses may be present in the isoflavone-supplemented dry feed at about 2 weight percent to about 4 weight percent, such as about 3.1 weight percent. Fiber may be present in the isoflavone-supplemented dry feed at about 2 weight percent to about 3 weight percent, such as about 2.5 weight percent. Isoflavone-supplemented dry feed may contain amino acids at about 4 weight percent to about 8 weight percent, such as about 5.7 weight percent. Isoflavone-supplemented dry feed may contain minerals, such as magnesium and calcium, at less than about 1 weight percent each. Moisture may be present in the dry feed at about 10 to 14 weight percent, such as about 13 weight percent.

In some implementations, the young animal may ingest the isoflavone-supplemented feed starting at about 1 to 5 days of age, about 1 to about 3 days of age, or about 1 to about 2 days of age and may consume the feed for the prescribed feeding period. For instance, the young animal may ingest the dry feed for one week during the first three weeks of life, for two weeks during the first three weeks of life, or over the course of the first three weeks of life, and beginning at any of the aforementioned ages or age ranges. In a particular example, the young animal may begin to ingest the dry feed at 3 days of age and may ingest the dry feed for three consecutive weeks until 24 days of age. In another example, the young animal may begin to ingest the dry feed at 1 to 5 days of age and may ingest the dry feed for two consecutive weeks over the next three-week period. Consequently, for instance, where the young animal begins to ingest the dry feed at day 5 of life, the three-week feeding period may end at day 26 of life.

In response to ingesting the isoflavone-supplemented dry feed, young poultry animals improve performance. Improved performance may include, but is not limited to improved weight gain, improved weight gain over a feeding period, improved feed efficiency, and improved feed efficiency over a feeding period. The improved performance may be realized after a feeding period. The improved performance may come without negatively affecting the animals' health.

In some particular examples, in response to ingesting the isoflavone-supplemented feed, weekly weights of animals receiving supplemental isoflavone may increase or increase significantly during at least a portion of a three-week feeding period beginning at about day one of life. Weekly gains by animals receiving supplemental isoflavone may increase or increase significantly for the three-week period compared to animals receiving the same feed without the supplemental isoflavone. Feed efficiency, or feed-to-gain ratio, may also be improved or improve significantly for this feeding period or a portion thereof. For example, total feed efficiency for animals receiving the isoflavone-supplemented feed may improve about 2.6% compared to animals receiving the same feed without the supplemental isoflavone. Health problems and death incidence for animals receiving the isoflavone-supplemented feed may be comparable to that of animals receiving the same feed without the supplemental isoflavone, demonstrating the health of the animal is not negatively affected by the inclusion of supplemental isoflavone in the diet.

### EXAMPLES

### Example 1—Supplemental Isoflavones in Chick Diet

Young, healthy chicks were studied to determine the effects of feeding isoflavones in the diet. Although benefits flowing from chicks ingesting isoflavone-supplement diets are disclosed in this Example, the Example should not be construed as limiting and other animals and feeding approaches disclosed herein may benefit from the feeding methods and systems of the Example.

Materials and Methods:

One hundred forty four day-old Cornish cross broiler chicks were assigned by weight to one of two dietary treatments: control and isoflavone-supplemented. Chicks were housed in 24 battery pens (12 per treatment) each containing six birds. Treatments were stratified within each row of the chick battery. Chicks were raised according to the Purina SOP for Young Poultry at the Purina Animal Nutrition Center in Eastern Missouri.

The control diet formulation is presented in Table 1 below.

TABLE 1
Control Diet Components

| Ingredient | Percent (By Weight) |
|---|---|
| Ground Corn (fine) | 52.7119 |
| Dehulled Soymeal | 25.7928 |
| Wheat Red Dog | 8.0824 |
| Gluten Meal 60 | 3.0000 |
| Ameri-Bond ® 2X (Borregaard | 2.5000 |
| LignoTech, Rothschild, WI) | |
| Molasses | 2.2420 |
| Calcium Carbonate | 1.2745 |
| Micro 1 (micronutrients) | 1.2500 |
| Phosphate 21 (mono dicalcium) | 1.0452 |
| Soy Oil (crude) | 1.0000 |
| L-Lysine | 0.5277 |
| Salt | 0.3173 |
| DL-Methionine | 0.2562 |

The test isoflavone (ISF) diet formulation was similar to the control diet but an isoflavone concentrate (NovaSoy 400, Archer Daniels Midland Co.) was added to provide 1500 ppm of added isoflavone (1500 mg isoflavone per 1 kg of feed). NovaSoy 400 is a 40% soy isoflavone product containing an aglycone ratio similar to that in soybeans. Calculated total isoflavone levels for the control diet was 890 mg/kg of feed and 2390 mg/kg of feed for the test supplemental isoflavone diet. Both diets were formulated to contain 20 weight percent protein, 3.5 weight percent fat, and 1.37 weight percent lysine.

Chicks were fed ad libitum amounts of the control or the supplemental isoflavone diet during the course of the trial. Body weights were collected each week of a three-week trial by weighing the entire pen of chicks. Feed was weighed weekly throughout the trial and feed-to-gain ratio was calculated. Animals were monitored for health issues, including leg lesions and death, throughout the trial.

Results:

Performance data for chicks fed these treatments is presented in Table 2.

TABLE 2
Chick Performance with Control vs. Isoflavone-Supplemented Feed

| | Treatment | | | |
|---|---|---|---|---|
| | | Test | | |
| Item | Control | (ISF 1500 ppm) | SEM | P-value |
| Start wt., g/hd | 39.69 | 39.87 | 0.2663 | 0.63 |
| Wk 1 wt., g/hd | 164.84 | 166.08 | 3.8008 | 0.82 |
| Wk 2 wt., g/hd | 420.58 | 426.34 | 7.8938 | 0.61 |
| Wk 3 wt., g/hd | 908.74 | 931.64 | 13.9526 | 0.25 |
| Wk 1 gain, g/hd | 125.15 | 126.21 | 3.6798 | 0.84 |
| Wk 2 gain, g/hd | 255.74 | 260.26 | 7.4038 | 0.67 |
| Wk 3 gain, g/hd | 488.16 | 505.30 | 8.281 | 0.15 |
| Total gain, g/hd | 869.05 | 891.77 | 13.9916 | 0.26 |
| Wk 1 intake, g/hd | 146.53 | 144.81 | 4.2527 | 0.77 |
| Wk 2 intake, g/hd | 383.20 | 388.07 | 10.236 | 0.74 |
| Wk 3 intake, g/hd | 772.49 | 769.17 | 14.7938 | 0.87 |
| Total intake, g/hd | 1302.22 | 1302.04 | 24.3825 | 0.99 |
| Wk 1 F:G, g | 1.173 | 1.146 | 0.01273 | 0.16 |
| Wk 2 F:G, g | 1.502 | 1.492 | 0.01867 | 0.71 |
| Wk 3 F:G, g | 1.583 | 1.525 | 0.02562 | 0.12 |
| Total F:G, g | 1.499 | 1.460 | 0.01533 | 0.09 |

Summary of Results:

Supplemental isoflavone added to a control chick diet significantly enhanced chick performance in this three-week evaluation. The bold numbers in Table 1 indicate the results are statistically significant, i.e., have a P-value of 0.20 or lower.

Weekly weights of chicks receiving supplemental isoflavone in chick feed increased compared to those receiving control feed during each week of the study. Weekly gain and total gain of chicks receiving supplemental isoflavone in chick feed increased compared to those receiving control feed during each week of the study. Weekly gain at Week 3 for chicks fed the isoflavone-supplemented diet was 2.6% greater, or nearly 23 grams heavier, than chicks fed the control feed. This represents a significant increase in weekly gain (P=0.15) and suggests further improvements might result over longer feeding periods.

Weight gain in chicks receiving supplemental isoflavone in chick feed was not due to increased intake, as weekly intake and total intake was nearly identical between the two groups (P=0.99 for total intake). Chicks receiving supplemental isoflavone gained more weight than chicks receiving control feed while consuming the same amount of food.

Weekly and total feed-to-gain ratio of chicks receiving supplemental isoflavone in chick feed improved compared to those receiving control feed during each week of the study. Weekly feed-to-gain ratio was significantly improved at Week 1 and Week 3 (P=0.16 and 0.12, respectively). Total feed efficiency in chicks fed the isoflavone-supplemented diet improved by 2.6% compared to chicks fed the control feed, which is a significant improvement (P=0.09). Weekly feed efficiency at Week 3 in chicks fed the isoflavone-supplemented diet improved by 3.6% compared to chicks fed the control feed, suggesting further improvements might result over longer feeding periods.

No health problems were observed for chicks in either group. Each group experienced a low death incidence.

These data indicate that isoflavone supplementation can enhance the performance of young poultry. Enhanced performance can be achieved without negative health effects.

Although the present disclosure provides references to preferred embodiments, persons skilled in the art will recognize that changes may be made in form and detail without departing from the spirit and scope of the invention.

1. A method of feeding a young poultry animal an isoflavone-supplemented feed comprising:
providing a feed to a young poultry animal for at least one week during the first three weeks of the young poultry animal's life during which the young poultry animal ingests the feed, the feed comprising a supplemental level of isoflavone added to the feed such that the total isoflavone level is about 1640 mg/kg of the feed to about 3890 mg/kg of the feed,
wherein the young poultry animal experiences improved performance in response to ingesting the isoflavone-supplemented feed for the at least one week.

2. The method of claim 1, wherein the young poultry animal is provided the feed for at least about three weeks and at least one week is during the first three weeks of the young poultry animal's life.

3. The method of claim 1, wherein a protein level in the feed is about 20 weight percent.

4. The method of claim 1, wherein isoflavone level of the feed prior to supplementation is about 890 mg/kg of the feed.

5. The method of claim 1, wherein the supplemental isoflavone level is about 750 mg/kg of the feed to about 3000 mg/kg of the feed.

6. The method of claim 5, wherein the supplemental isoflavone level is about 1500 mg/kg of the feed.

7. The method of claim 1, wherein the improved performance experienced by the young poultry animal is one or both of an improved weight gain or an improved feed-to-gain ratio.

8. The method of claim 1, wherein the young poultry animal is a broiler chick.

9. The method of claim 1, wherein the isoflavone supplement is an isoflavone isolate derived from soy.

10. The method of claim 9, wherein the isoflavone supplement comprises isoflavone aglycone.

11. The method of claim 10, wherein the isoflavone aglycone comprises genistein aglycone.

12. A method of improving performance of a young poultry animal comprising:
providing a feed to a young poultry animal for at least one week during the first three weeks of the young poultry animal's life during which the animal ingests the feed, the feed comprising a supplemental level of isoflavone added to the feed such that the total isoflavone level is about 1640 mg/kg of the feed to about 3890 mg/kg of the feed,
wherein in response to ingesting the isoflavone-supplemented feed for the at least one week, the young poultry animal experiences improved performance compared to a young poultry animal that ingests the same feed without supplemental isoflavone.

13. The method of claim 12, wherein the improved performance experienced by the young poultry animal is improved weight gain.

14. The method of claim 12, wherein the improved performance experienced by the young poultry animal is improved feed-to-gain ratio.

15. The method of claim 12, wherein the young poultry animal is provided the feed for at least about three weeks and at least one week is during the first three weeks of the young poultry animal's life.

16. The method of claim 12, wherein a protein level in the feed is about 20 weight percent.

17. The method of claim 12, wherein isoflavone level of the feed prior to supplementation is about 890 mg/kg of the feed.

18. The method of claim 12, wherein the supplemental isoflavone level is about 750 mg/kg of the feed to about 3000 mg/kg of the feed.

19. The method of claim 12, wherein the young poultry animal is a broiler chick.
